(19) United States
(12) Patent Application Publication
(10) Pub. No.: US 2024/0423917 A1
(43) Pub. Date: Dec. 26, 2024

(54) USE OF NANOPARTICLES FOR THE TREATMENT OF FISTULIZING ANOPERINEAL LESIONS

(71) Applicants: INSERM (INSTITUT NATIONAL DE LA SANTÉ ET DE LA RECHERCHE MÉDICALE), Paris (FR); UNIVERSITÉ PARIS 13 PARIS NORD, Villetaneuse (FR); UNIVERSITÉ PARIS CITÉ, Paris (FR); ASSISTANCE PUBLIQUE-HÔPITAUX DE PARIS (APHP), Paris (FR)

(72) Inventors: Didier LETOURNEUR, Paris (FR); Antoine CAZELLES, Paris Cedex 18 (FR); Eric OGIER-DENIS, Rennes (FR); Xavier TRETON, Paris Cedex 18 (FR); Teresa SIMON YARZA, Paris (FR); Yoann LALATONNE, Paris (FR); Laurence MOTTE, Paris (FR)

(21) Appl. No.: 18/687,505

(22) PCT Filed: Sep. 2, 2022
(86) PCT No.: PCT/EP2022/074429

§ 371 Date: Feb. 28, 2024

(30) Foreign Application Priority Data
Sep. 3, 2021 (EP) 21306204.5

(51) Int. Cl. A61K 9/14; A61K 9/00; A61K 33/00; A61K 33/26; A61K 33/32; A61K 33/34; A61K 45/06; A61P 1/00; B82Y 5/00

(52) U.S. Cl. CPC A61K 9/14; A61K 9/0019; A61K 33/00; A61K 33/26; A61K 33/32; A61K 33/34; A61K 45/06; A61P 1/00; B82Y 5/00

(57) ABSTRACT

Anoperineal lesions (APLs) are frequent in Crohn's disease and are particularly difficult to treat because of the induced tissue destruction and their recurrence. Now, the inventors developed the first preclinical model of perianal fistula with pathological inflammation of the rectum that allows to test and optimize new treatments. Then the aim of the inventors was to perform a preclinical study using a solution of iron oxide nanoparticles for treatment of perianal fistula in a rat model of perianal fistulizing Crohn's disease. They showed that all treated inflammatory fistulas were permanently filled or closed with the solution of iron oxide nanoparticles (from day 1 to day 7). Accordingly, use of nanoparticles seems to be a promising new treatment of perianal fistulizing Crohn's disease.

### FIELD OF THE INVENTION

The present invention is in the field of medicine, in particular gastroenterology.

### BACKGROUND OF THE INVENTION

Crohn's disease (CD) belongs to the group of inflammatory bowel diseases (IBD), which main inflammatory sites are the small intestine, colon, anus, and perineum. Anoperineal lesions (APLs) are frequent and particularly difficult to treat because of the induced tissue destruction and their recurrence. APLs are classified as non-fistulizing lesions (skin tags, fissures, ulcers, anorectal stricture and anal cancer) and fistulizing lesions (simple or complex perineal fistulas, rectovaginal fistulas +/−abscessed) [1]. Fistulizing anoperineal lesions (FAPLs) are the most severe ones, with a long-term risk of anal sphincter destruction, incontinence, permanent stoma and anal cancer. FAPLs' management is complex and multimodal, requiring the complementary expertise of gastroenterologists, radiologists, and surgeons. More than two-thirds of patients have an abscess associated with their FAPLs so the treatment first includes a surgical phase of examination, abscess drainage and insertion of a seton through the fistula tract. The second phase is medical treatment, using TNF alpha antagonists in order to control disease-related inflammation. A final surgical phase aiming to close the fistula tract may be considered in a symptomatic patient with no concomitant abscess and with medically controlled proctitis [2]. Several surgical procedures are available to close the fistula tract: fibrin glue, chronic seton, endorectal advancement flaps, plug, LIFT, and VAAFT [2,3]. FAPLs relapse in about two-thirds of patients leading to a repetition of surgical procedures, including for patients with uncontrolled disease, diverting stoma or proctectomy. Recently, local injection of mesenchymal stem cells has shown interesting and promising results with regard to fistula closure [4,5]. However, there are many limitations in cell-based therapy: type of stem cells, tissue origin, cells' concentration, fate of stem cells, logistic coordination between the production site and hospital, and availability of treatments. Therefore, new and more controllable treatments are expected.

Recently, a promising approach to regenerative medicine using iron oxide nanoparticles has been proposed [7]. The authors demonstrate that nanobridging (adhesion by aqueous nanoparticle solutions) can be used in vivo to achieve rapid and strong closure and healing of deep wounds in skin and liver.

### SUMMARY OF THE INVENTION

The present invention is defined by the claims. In particular, the present invention relates to use of nanoparticles for the treatment of fistulizing anoperineal lesions.

### DETAILED DESCRIPTION OF THE INVENTION

The inventors developed the first preclinical model of perianal fistula with pathological inflammation of the rectum that allows to test and optimize new treatments. Then the aim of the inventors was to perform a preclinical study using a solution of iron oxide nanoparticles for treatment of perianal fistula in a rat model of perianal fistulizing Crohn's disease. They showed that all treated inflammatory fistulas were permanently filled or closed with the solution of iron oxide nanoparticles (from day 1 to day 7). Accordingly, use of nanoparticles seems to be a promising new treatment of perianal fistulizing Crohn's disease.

Accordingly, the first object of the present invention relates to a method of treating a fistulizing anoperineal lesion in patient in need thereof comprising injecting in the fistula tract a solution comprising an amount of nanoparticles.

As used herein, the term “subject”, “individual” or “patient” is used interchangeably and refers to any subject for whom diagnosis, treatment, or therapy is desired, particularly humans. Other subjects may include cattle, dogs, cats, guinea pigs, rabbits, rats, mice, horses, and the like. In some preferred embodiments, the subject is a human.

As used herein, the term “fistulizing anoperineal lesion” has its general meaning in the art and refers to an abnormal connection between the anal canal and the perianal skin.

As used herein, the term “treating” refers to repairing a fistula, as well as preventing a fistula from worsening or recurring.

In some embodiments, the patient suffers from an inflammatory bowel disease, and more particularly from Crohn's disease.

As used herein the term “inflammatory bowel disease” has its general meaning in the art and refers to any inflammatory disease that affects the bowel. The term includes but is not limited to ulcerative colitis, Crohn's disease, especially Crohn's disease in a state that affect specifically the colon with or without ileitis, microscopic colitis (lymphocytic colitis and collagenous colitis), infectious colitis caused by bacteria or by virus, radiation colitis, ischemic colitis, pediatric colitis, undetermined colitis, and functional bowel disorders (described symptoms without evident anatomical abnormalities).

As used herein, the term “nanoparticles” means particles from 1 nm to 1000 nm, preferably from 2 to 500 nm and even more preferably from 5 to 300 nm in size. For most nanoparticles, the size of the nanoparticles is the distance between the two most distant points in the nanoparticle. For anisotropic nanoparticles, such as tubes whiskers or cylinders, the size of the diameter is the diameter of the smallest cylinder in which the nanoparticle is inscribed. Nanoparticle size can be determined by different methods such as Dynamic Light Scattering (DLS), Small Angle X-ray Scattering (SAXS), Scanning Mobility Particle Sizer (SMPS), Scanning Electron Microscopy (SEM), Transmission Electron Microscopy (TEM) (Orts-Gil, G., K. Natte, et al. (2011), Journal of Nanoparticle Research 13 (4): 1593-1604; Alexandridis, P. and B. Lindman (2000), Amphiphilic Block Copolymers: Self-Assembly and Applications, Elsevier Science; Hunter, R. J. and L. R. White (1987). Foundations of colloid science, Clarendon Press.).

In some embodiments, the nanoparticles of the present invention have a size of about 5, 10, 15 or 20 nm.

As used herein, the term “about” is used to denote a degree of variability in a value or range, for example, within 10%, within 5%, or within 1% of a stated value or of a stated limit of a range.

In some embodiments, the nanoparticles of the present invention are made of different chemical nature, of different sizes, and/or of different shapes. In some embodiments, the nanoparticles can be in the form of a sphere, needle, flake, platelet, tube, fiber, cube, prism, whiskers or have an irregular shape.

In some embodiments, the nanoparticles are selected among solid nanoparticles. In some embodiments, nanoparticles can be mineral, organic or mixed, and be coated or grafted.

In some embodiments, the nanoparticles are mineral nanoparticles. Among the inorganic nanoparticles, one can mention metal oxides, alumina, silica, kaolin, hydroxyapatite, calcium carbonate, silicates such as micas quartz, zeolites or clays such as hectorite, laponite, montmorillonite, bentonite, smectite. Mineral particles may include, but are not limited to, metal particles. Metal particles encompass particles formed exclusively with metallic alloys or metals chosen among alkaline earth metal, transitional metal, rare earth metal, and alloys thereof. In some embodiments, the metal may be aluminum, copper, cadmium, selenium, silver, gold, indium, iron, platinum, nickel, molybdenum, silicon, titanium, tungsten, antimony, palladium, zinc, tin, and alloys thereof. These metal particles may be metal organo modified nanoparticles having chemical entities grafted to their surface or having a self-assembled monolayer of compounds, such as organosulfur compounds, on their surface.

In some embodiments, the nanoparticles are made of metal oxides, such as iron oxides (FeO, Fe₂O₃, Fe₃O₄), cerium oxide (CeO), alumina (Al₂O₃), zirconium oxide (ZrO₂), titanium oxide (TiO₂), titanates (BaTiO₃, Ba0.5Sr0.5TiO₃, SrTiO₃), indium oxide (In₂O₃), tin oxide (SnO₂), antimony oxide (Sb₂O₃), magnesium oxide (MgO), calcium oxide (CaO), manganese oxides (Mn₃O₄, MnO₂), molybdenum oxide (MoO₃), silica (SiO₂), zinc oxide (ZnO), yttrium oxide (Y₂O₃), bismuth oxychloride, Copper oxides (CuO, Cu₂O).

In some embodiments, the nanoparticles are made of metal oxides, wherein metal oxides are selected from the group consisting of iron oxides (FeO, Fe₂O₃, Fe₃O₄), cerium oxide (CeO), alumina (Al₂O₃), zirconium oxide (ZrO₂), titanium oxide (TiO₂), titanates (BaTiO₃, Ba0.5Sr0.5TiO₃, SrTiO₃), indium oxide (In₂O₃), tin oxide (SnO₂), antimony oxide (Sb₂O₃), magnesium oxide (MgO), calcium oxide (CaO), manganese oxides (Mn₃O₄, MnO₂), molybdenum oxide (MoO₃), silica (SiO₂), zinc oxide (ZnO), yttrium oxide (Y₂O₃), bismuth oxychloride, and/or Copper oxides (CuO, Cu₂O).

In some embodiments, the nanoparticles will act as contrast agents that can be directly imaged. In particular, the nanoparticles of the present invention allow the possibility for a structural or functional imaging procedure, e.g. for following the implantation of the nanoparticles so as to verify that the maintenance of the closing of the fistula. Accordingly the nanoparticles can be detectable by imaging techniques such as ultrasonography, elastography, Supersonic Shear Wave Imaging, Magnetic Resonance Imaging (MRI), Positron Emission Tomography (PET), Single Photon Emission Computed Tomography (SPECT), fluorescence spectroscopy, Computed Tomography, X-ray radiography, or any combination of these techniques.

In some embodiments, the nanoparticles are designed to be detectable by Magnetic Resonance Imaging (MRI). MRI, which is an application of Nuclear Magnetic Resonance (NMR), has evolved into one of the most powerful non-invasive techniques in diagnostic clinical medicine and biomedical research. MRI has the advantage (over other high-quality imaging methods) of not relying on potentially harmful ionizing radiation.

In some embodiments, the nanoparticles consist of ultrasmall superparamagnetic iron oxide (USPIO) particles. USPIO particles are currently under investigation as contrast agents for imaging human pathologies (C. Corot et al., Adv. Drug Deliv. Rev., 2006, 56:1472-1504). They are composed of a crystalline iron oxide core containing thousands of iron atoms which provide a large disturbance of the Magnetic Resonance signal of surrounding water. In contrast to other types of nanoparticles such as quantum dots (currently under investigation as extremely sensitive fluorescent probes), USPIO particles exhibit a very good biocompatibility. Chemical coating of USPIO particles is required to ensure their dispersion in biological media. Polysaccharides, such as dextran and its carboxymethylated derivatives, are currently used as coatings. USPIO particles are known in the art and have been described (see, for example, J. Petersein et al., Magn. Reson. Imaging Clin. Am., 1996, 4:53-60; B. Bonnemain, J. Drug Target, 1998, 6:167-174; E. X. Wu et al., NMR Biomed., 2004, 17:478-483; C. Corot et al., Adv. Drug Deliv. Rev., 2006, 58:1471-1504; M. Di Marco et al., Int. J. Nanomedicine, 2007, 2:609-622). USPIO particles are commercially available, for example, from AMAG Pharmaceuticals, Inc. under the tradenames Sinerem® and Combidex®.

In some embodiments, the nanoparticles of the present invention are iron oxide Fe3O4 Ultrasmall Superparamagnetic Iron Oxide (USPIO) nanoparticles as described in the EXAMPLE.

The particles of the present invention are prepared according to any conventional method known in the art. For instance, the iron oxide Fe3O4 Ultrasmall Superparamagnetic Iron Oxide (USPIO) nanoparticles are prepared as described in the EXAMPLE.

In some embodiments, the nanoparticles are injected in the fistula tract as an aqueous solution of nanoparticles.

As used herein, the term “aqueous solution” has its general meaning in the art and refers to a solution in which the solvent is water.

Aqueous solutions of nanoparticles are commercially available. One can mention the aqueous solutions of colloidal silica Ludox® from Grace Davison. They can be prepared for any of the above-mentioned material by using methods known to the skilled professional Stöber et al. method (Controlled growth of monodisperse silica spheres in the micron size range, Journal of colloid and interface science (1968)).

In some embodiments, the nanoparticles are injected in the fistula tract as an aqueous alcohol solution of nanoparticles.

As used herein, the term “aqueous alcohol solution” has its general meaning in the art and refers to a mixed solution of water and an alcohol, for instance, a solution obtained by dissolving 1 to 300 parts by weight of a primary alcohol such as ethanol or methanol in 100 parts by weight of water.

In some embodiments, the aqueous solution of nanoparticles consists essentially of nanoparticles suspended in water. It means that other components can be present in the suspension, but they do not modify the properties of the suspension in a noticeable manner. Especially, other components can be present in the suspension, but they have to be selected so as not to modify the adhesive properties of the suspension (e.g. dispersion property of the nanoparticles).

According to the present invention, the solution (such as aqueous solution) of nanoparticles which can be used according to the invention does not contain any other adhesive agent. It means that the solution of nanoparticles does not contain a compound known as an adhering agent in a concentration that would allow it to play the function of adhesive agent. Among known adhesive agents, one can mention synthetic adhesives such as monomers, synthetic polymers (other than polymer nanoparticles), notably cyanoacrylates, urethanes, dendrimers; or natural adhesives such as fibrin, collagen, gelatin, and polysaccharides.

According to the invention, the nanoparticles have the function of adhering agent in the compositions wherein they are present. Typically, in the preparations as above described, the nanoparticles represent from 10 to 100% by weight of the weight of the dry matter of the solution. Typically, the nanoparticles represent from 20 to 100% by weight of the weight of the dry matter of the preparation, (e.g. the aqueous solution), even more preferably, from 30 to 100%, and advantageously, from 40 to 100%, better from 50 to 100%, even better from 60 to 100%, preferably from 70 to 100%, even better from 80 to 100%, even more preferably from 90 to 100%. According to a particular embodiment, the nanoparticles represent from 95 to 100% by weight of the weight of the dry matter of the solution (such as aqueous solution), even better from 98 to 100%, and even more preferably from 99 to 100%.

In some embodiments, the concentration of the nanoparticles, in particular, the iron oxide Fe3O4 Ultrasmall Superparamagnetic Iron Oxide nanoparticles is typically comprised between 10 mg/ml and 1000 mg/ml. In some embodiments, the concentration of the nanoparticles in the solution (such as aqueous solution) is comprised between 50 mg/ml and 250 mg/ml. In some embodiments, the concentration is about 10, 20, 30, 50, 60, 70, 80 90, 100, 110, 120, 130, 140, 150, 160, 170, 180, 190, 200, 210, 220, 230, 240, 250, 300, 350, 400, 450, 500, 550, 600, 650, 700, 800, 900 or 1000 mg/ml.

Typically, the injection of the solution of nanoparticles of the invention is applied using conventional techniques, such as for instance a syringe.

The quantity of nanoparticles deposited at the surface of the fistula surface is from 0.1 mg/m² to 10 g/m². Depending on the size of the nanoparticles, the coverage of the surface is to be adjusted. These values can be from 1 mg/m^{2,} preferably for small particles, and up to 0.2 g/m², preferably for large particles. For large particles (typically of the order of 300 nm) the coverage is large, of the order of 4 g/m². For particles of smaller size (diameter of about 2 nm) rates coverage is preferably of the order of 10 mg/m². In particular, it is believed that optimum adhesion is obtained for a dense monolayer on the nanoparticles surface. The density of coverage can be evaluated on the assembly by ATR-FTIR or by SEM.

In some embodiments, the volume of nanoparticles that is deposited at the surface ranges from 0.01 to 5 μl. per mm². Typically, a volume of 0.1; 0.2; 0.3; 0.4; 0.5; 0.6; 0.7; 0.8; 0.9; 1; 1.1; 1.2; 1.3; 1.4; 1.5; 1.6; 1.7; 1.8; 1.9; 2; 2.1; 2.2; 2.3; 2.4; 2.5; 2.6; 2.7; 2.8; 2.9; 3; 3.1; 3.2; 3.3; 3.4; 3.5; 3.6; 3.7; 3.8; 3.9; 4; 4.1; 4.2; 4.3; 4.4; 4.5; 4.6; 4.7; 4.8; 4.9; or 5 μl per mm² is deposited at the surface of the fistula tract.

In some embodiments, once the solution (such as aqueous solution) of nanoparticles is injected in the fistula tract, a pressure is applied on the surface where the fistula tract arises. In some embodiments, a pressure on the peritoneum is applied. In some embodiments, the pressure is applied for a time of at least, 1, 2, 3, 4, 5, 6, 7, 8, 9 10, 11, 12, 13, 14, 15, 16, 17 18, 19 20; 21; 22; 23; 24; 25; 26; 27; 28; 29; 30; 31; 32; 33; 34; 35; 36; 37; 38; 39; 40; 41; 42; 43; 44; 45; 46; 47; 48; 49; 50; 51; 52; 53; 54; 55; 56; 57; 58; 59; 60; 61; 62; 63; 64; 65; 66; 67; 68; 69; 70; 71; 72; 73; 74; 75; 76; 77; 78; 79; 80; 81; 82; 83; 84; 85; 86; 87; 88; 89; 90; 91; 92; 93; 94; 95; 96; 97; 98; 99; 100; 101; 102; 103; 104; 105; 106; 107; 108; 109; 110; 111; 112; 113; 114; 115; 116; 117; 118; 119; 120; 121; 122; 123; 124; 125; 126; 127; 128; 129; 130; 131; 132; 133134 135 136 137 138 139 140 141 142 143 144 145 146 147 148 149 150 151 152 153 154 155 156 157 158 159 160 161 162 163 164 165 166 167 168 169 170 171 172 173 174 175 176 177 178 179 180 181 182 183 184 185 186 187 188 189 190 191 192 193 194 195 196 197 198 199 200 201 202 203 204 205 206 207 208 209 210 211 212 213 214 215 216 217 218 219 220 221 222 223 224 225 226 227 228 229 230 231 232 233 234 235 236 237 238 239 240 241 242 243 244 245 246 247 248 249 250 251 252 253 254 255 256 257 258 259 260 261 262 263 264 265 266 267 268 269 270 271 272 273 274 275 276 277 278 279 280 281 282 283 284 285 286 287 288 289 290 291 292 293 294 295 296 297 298 299 or 300s. In some embodiments, the pressure is applied for about one minute. In some embodiments, the pressure is a manual pressure (e.g. with a finger), a mechanical pressure, a suture pressure, or a staple pressure.

The method described above may further comprise administering a therapeutic agent to the patient being treated, e.g. systemically or locally at the site of the fistulizing anoperineal lesion. In some embodiments, the therapeutic agent is administered separately, e.g. simultaneously with the method of the present invention, before the method is performed, or after the method is performed.

In some embodiments, the therapeutic agents are suitable for the treatment of Crohn's disease. Exemplary Crohn's disease therapeutic agents are anti-inflammatory agents such as agents comprising mesalamine, immunosuppressive agents such as 6-mercaptopurine and azathioprine, antibiotics, and antidiarrheal agents such as diphenoxylate, loperamide, and codeine.

In some embodiments, the therapeutic agent is a TNFα blocking agent.

As used herein, the term “TNFα blocking agent” or “TBA”, it is herein meant a biological agent which is capable of neutralizing the effects of TNFα. Said agent is a preferentially a protein such as a soluble TNFα receptor, e.g. Pegsunercept, or an antibody. In some embodiments, the TBA is a monoclonal antibody having specificity for TNFα or for TNFα receptor. In some embodiments, the TBA is selected in the group consisting of Etanercept (Enbrel®), Infliximab (Remicade®), Adalimumab (Humira®), Certolizumab pegol (Cimzia®), and golimumab (Simponi®). Recombinant TNF-receptor based proteins have also been developed (e.g. etanercept, a recombinant fusion protein consisting of two extracellular parts of soluble TNFα receptor 2 (p75) joined by the Fc fragment of a human IgG1 molecule). A pegylated soluble TNF type 1 receptor can also be used as a TNF blocking agent. Additionally, thalidomide has been demonstrated to be a potent inhibitor of TNF production. TNFα blocking agents thus further include phosphodiesterase 4 (IV) inhibitor thalidomide analogues and other phosphodiesterase IV inhibitors. As used herein, the term “etanercept” or “ETA” denotes the tumor necrosis factor-alpha (TNFα) antagonist used for the treatment of rheumatoid arthritis. The term “etanercept” (ETA, ETN, Enbrel) is a recombinant TNF-receptor IgG-Fc-fusion protein composed of the p75 TNF receptor genetically fused to the Fc domain of IgG1. Etanercept neutralizes the proinflammatory cytokine tumor necrosis factor-α (TNFα) and lymphotoxin-α (Batycka-Baran et al., 2012).

The invention will be further illustrated by the following figures and examples. However, these examples and figures should not be interpreted in any way as limiting the scope of the present invention.

### FIGURES

FIG. 1: Study timeline and main steps for inflammatory fistulas formation.

FIG. 2: Physico-chemical characterisations of USPIO@Citrate nanoparticles. a) TEM image, b) Magnetization curve at room temperature, c) FTIR spectra of coated NP and corresponding coating molecule.

FIG. 3: Follow-up after treatment.

### EXAMPLE

### Material & Methods

### Study Design and Animals

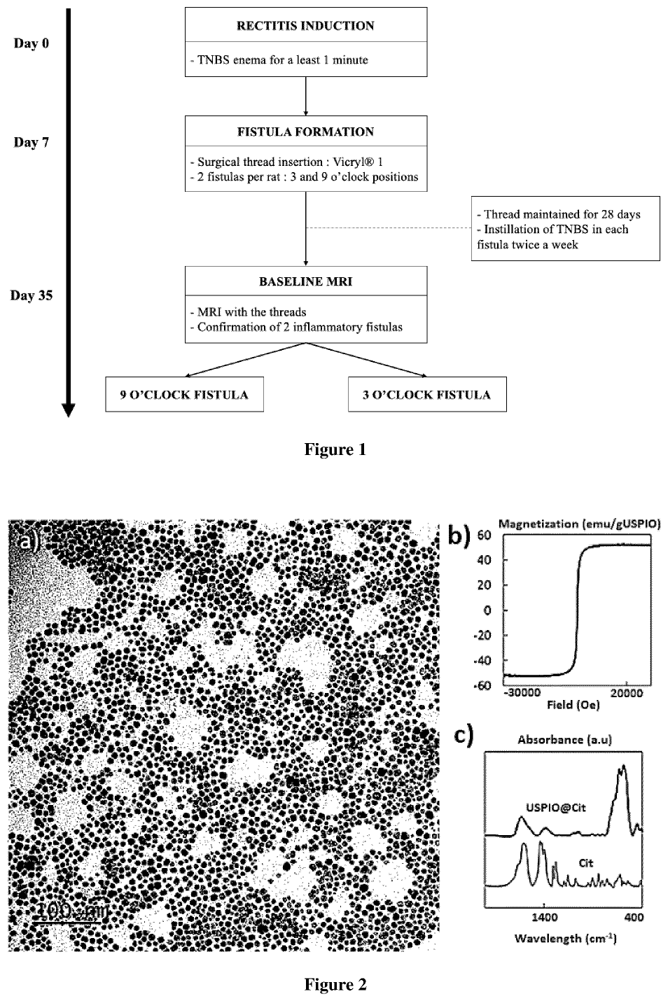

A preclinical study testing USPIO for treatment of perianal fistula was performed in a rat model of perianal fistulizing Crohn's disease developed in our laboratory [6]. This experimental model develops perianal fistula with pathological inflammation of the rectum, characterized by a persistent fistula tract after more than 7 days. Two fistulas by rat were generated in a group of rats allowing that each rat was its own control. Main steps of inflammatory fistula formation are summarized in FIG. 1. Animals were male Sprague Dawley rats aged between 42 and 90 days. They were bred and housed in a conventional area, fed with irradiated food and hydrated orally ad libitum, including the day before and the day of the procedure. Up to two rats were housed in the same cage. Rats were weighed on the day of each procedure and then every 7 days. All experiments were performed in compliance with the European Community guidelines and approved by the Institutional Animal Care and Use Committee (no. APAFIS #23031-2019112613522589v5 Paris Nord ethic committee and French Research Ministry).

All procedures were performed according to the same anesthetic and analgesic protocol. Animals were anaesthetized with isoflurane inhalation (Isoflurane 3 L/min+O₂ 2 L/min during induction then Isoflurane 1.5 L/min+O₂ 2 L/min). Analgesia was performed by local injection of 20% Lidocaine Hydrochloride (1 mg/kg) and by subcutaneous abdominal injection of Buprenorphine (0.05 mg/kg/rat). Post-operatively, if the animal shows signs of pain-related stress, the analgesic used was Buprenorphine at the same dosage.

### Preclinical Model of Perianal Fistulizing Crohn's Disease

Day 0, RECTITIS INDUCTION: rectitis was induced with a 500 μL rectal enema containing a TNBS solution (2.4.6-trinitrobenzenesulfonic acid solution, Picrylsulfonic acid, SIGMA laboratory). The TNBS solution contained 85.3 μL of TNBS (i.e. 25 mg), 250 μL of 100% ethanol and 164.7 μL of saline solution. The rectal enema was maintained for at least 1 minute. The rectal inflammatory peak was reached between day 5 and day 7 after the enema.
Day 7, FISTULA FORMATION: at the peak of the rectal inflammation, 2 fistulas were created on each rat. The rats were placed in a supine position and this position was the reference to locate the fistulas (scrotum at 12 o'clock, tail at 6 o'clock). Fistulas were created at 3 o′clock and 9 o'clock by inserting a surgical thread (Vicryl® 1, ETHICON Laboratory) into the rectum (internal orifice) and exiting at the perineum about 1 cm from the anal margin (external orifice). In order to obtain a good caliber, the fistula tract was sharpened with the surgical thread. A 18 G blunt fill needle was passed through the fistula as well as a 10 μL filter tips. We obtained internal and external orifices of approximately 2 mm in diameter. At the end of the surgical procedure, the thread was retained in the fistula tract and 100 μL of a TNBS solution was instilled within each tract. The TNBS solution contained, for a 100 μL instillation, 17.06 μL of TNBS (i.e. 5 mg), 50 μL of pure ethanol and 32.94 μL of saline solution. This concentration was the same concentration as the rectal enema.
Day 8 to day 34, MONITORING THREADS IN POSITION: the threads were maintained for 28 days. During this period, the rats were examined every 1 to 2 days to ensure that the fistulas were well tolerated and that no threads fell out. If threads were lost, they were reinserted the same day during a short anesthesia. Twice a week, 100 μL of the same TNBS solution was instilled within each fistula tract.
Day 35, BASELINE MRI: after maintaining threads for 28 days, 2 inflammatory fistulas were obtained and a perineal MRI was performed to assess the pre-treatment tracts.

### Treatment With an Aqueous Solution of Iron Oxide Nanoparticles

USPIO was composed with iron oxide Fe₃O₄ Ultrasmall Superparamagnetic Iron Oxide nanoparticles (USPIO-NP). USPIO-NP were synthesized using microwave non-aqueous sol-gel method [12]. The iron oxide nanoparticles surface has to be functionalized in order to obtain stability and bio-compatibility within physiological conditions. Herein, the USPIO-NP surface was functionalized with citrate ligands leading to stable USPIO suspension at pH 7 and high concentration (30 wt. %). The magnetic core size, measured by TEM, is equalled to 9.0±2.2 nm (FIG. 2a) and present a superparamagnetic behaviour with a saturate magnetization of 51±2 emu/guspio leading to strong T2 MRI contrast agent [12]. Citrate coating was qualitatively and quantitatively assessed by Fourier Transformed Infrared (FTIR) measurements (FIGS. 2b,c) and thermogravimetric analysis leading to 350 citrate molecules per nanoparticle.

After the baseline MRI (Day 35), surgical threads were removed and the fistula tract to be treated was randomly drawn. 2 μL of USPIO solution at 300 mg/ml was injected directly into the fistula tract. In order to close the tract, external pressure on the perineum was maintained for 1 minute. The control fistula tract was treated with 2 μL of a saline solution.

### Follow-Up and Fistula Assessment Methods

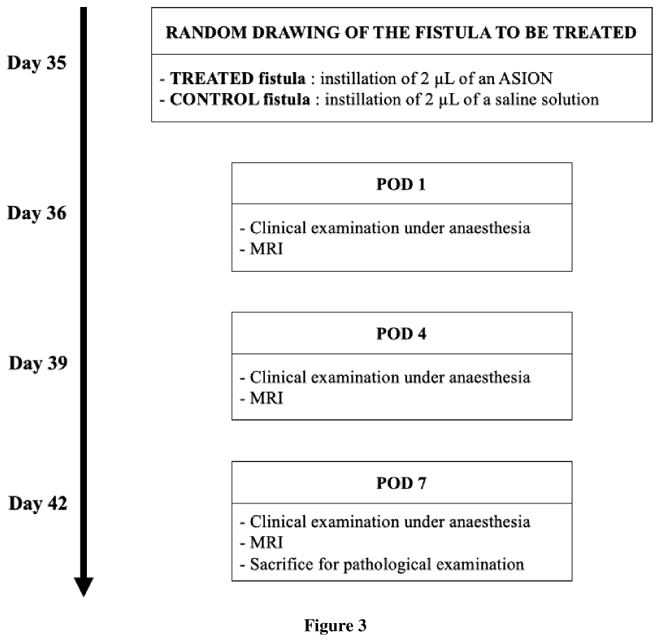

Follow-up after treatment is summarized in FIG. 3. Rats were assessed at post-operative day 1 (POD), POD 4 and POD 7. For each assessment, rats were clinically examined under general anesthesia and underwent a perineal MRI.

The main points of clinical examination were defined according to the CDAI and PDAI scores: behaviour, appearance, rectal bleeding, stool consistency, body weight and weight loss, and swelling/induration/infiltration of the perineum. The clinical diagnosis of fistula was defined by the presence of an external orifice and/or an internal orifice. The diagnosis of rectitis was defined by the presence of ano-rectal inflammation and/or bloody diarrhea.

Perineal MRI was performed on a 7T MRI system for small animals (Bio Spec, Bruker BioSpin, Ettlingen, Germany). The animal anesthetized with isoflurane was placed in an MRI cradle in a prone position, with the legs inserted first into the tunnel. The isocenter was placed on the presumed location of the external orifice of the fistula (at about 0.5 cm from the anus). Morphological axial T1-and T2-weighted images were acquired with fat saturation, covering 30 mm starting from the anus. Axial ultra-short echo time (UTE) images were acquired on the same region. Finally, diffusion-weighted imaging (DWI) was performed along 3 orthogonal directions, with different b values (0, 150, 400 and 800 s/mm²) and fat saturation to acquire 11 slices centered on the fistula track. Acquisition parameters are detailed in Table 1. Image interpretation and parameter measurements were performed by an abdominal radiologist (14 years of experience in IBD) and a research engineer (16 years of experience in pre-clinical MRI) who were blinded to the fistula group and to the pathological results. Each rat underwent 4 MRI sessions: the first session was performed before administration of the treatment. The other sessions were performed at day 1, 4 and 7 after treatment. The T2-weighted images from the first session were used to calculate a global proctitis score (GPS). Briefly, inflammation location and extent, seen as a hyperintense signal on T2-weighted images, were evaluated by the radiologist. The persistence of the residual fistula after surgical thread removal was assessed during each post-treatment MRI session based on its visibility (percentage of tract seen by the radiologist), its maximal diameter (mm), and the presence of an external and internal orifice compared with the same parameters evaluated at the time of the first MRI session. This assessment was performed using the T2-weighted images in priority, and completed if necessary using the UTE images (i.e. blurry area on the T2-weighted image, susceptibility artefact due to the iron nanoparticles present in the administered treatment). Tissue changes associated with the fistula tract were assessed by measuring the signal intensity on T2-weighted images (arbitrary units [a.u.], normalized to the muscle signal intensity) and the apparent diffusion coefficient (ADC, 10⁻³ mm²/s). Areas of interest were drawn directly around the tract on 3 consecutive slices, on the T2-weighted image, or on the b0 DW image. For DWI, the trace image was calculated for each b value, and then the ADC map was calculated using a pixel-by-pixel manner with monoexponential depiction of the signal intensity.

At POD 7 after clinical examination and MRI under general anesthesia, all rats were euthanized by cardiac explantation during sternotomy. To obtain a monobloc resection of the anorectal junction with the fistula tracts, an abdominoperineal resection was performed through a perineal approach. Dissection was done from bottom to top with a circumferential section of the perineum carrying the fistula tracts. The rectum was sectioned about 3 cm from the anus at the pelvic floor muscles. The operating piece was cut in half to separate the fistulas. Tissue samples were fixed for 24 hours in formaldehyde (Formal Solution, neutral buffered, 10%, Sigma laboratory) and stored at 4° C. Specimens were then accurately oriented and included in paraffin. Several μm-thick sections were cut with a microtome along the fistula tract. For each sample, 1 section was stained with hematein eosin, and 5 sections were prepared for immunohistochemical analyses. The diagnosis of fistula tract was based on the presence of the following histological criteria: a tract with a lumen from an internal orifice on the rectal mucosa to an external orifice on the perineal skin, pathological signs of proctitis (B and T lymphocytes, neutrophils, macrophages, granulomas), and the nature of the fistula tract was defined based on the presence or the absence of fibrosis, epithelialization, and acute or chronic inflammation.

### Outcomes

Primary outcome was the filling or closure of the fistula tract including the external or internal openings by the ASION evaluated by clinical examination, MRI and confirmed by pathological examination. Secondary outcomes were: evolution of fistula tracts' inflammation, model reassessment through the analysis of the controlled fistulas and safety of the ASION.

### Statistical Analysis

Quantitative data are expressed as a mean +standard deviation (range). Qualitative data are reported as frequency and percentage.

### Results

The preclinical study was 42 days long. We studied 20 rats and made 2 fistulas for each rat. All rats had soft stools and perineum tumefaction on day 7 after TNBS enema. While threads were maintained for 28 days, all rats were anaesthetized 8 times in average (min 6-max 11), in order to ensure threads were present and twice a week to instill 100 μL of TNBS solution in each fistula tract. New threads were reinserted 6 times in average during this period (min 0-max 11). Only 1 rat kept the same thread for 28 days. 12 rats developed an abscess during this phase which healed in about 1 week. Still during this phase, 3 rats tore out 1 of the 2 threads causing a fistulotomy. Those 3 rats with only 1 fistula tract were excluded from global analysis. 2 of them were treated with USPIO and 1 by a saline solution. They were kept alive until POD 14 in order to assess long-term fate of the USPIO suspension. Fistulas' characteristics before treatment of the 17 rats composing main analysis are summarized in Table 2. All rats presented, on clinical and radiological assessment, 2 inflammatory fistulas before treatment. 15 rats had a chronic rectitis graded at 1 and 2 rats had no rectitis before treatment. Mean MRI-calculated diameter was 2.2 mm (±0.6 [1.6-3.3]) for treated fistulas and 1.9 mm (±0.3 [1.5-2.3]) for control fistulas. Similar results were observed for the 3 excluded rats as detailed in Table 5.

### Control Fistulas

As presented in Tables 3 and 4, control fistulas had a low and poor natural healing process over time. At POD 7, only 4 tract had an internal orifice closed. 12 fistulas were still visualized on more than 50% of their tract. A total of 6 of the 7 control fistulas were still visible or had an open internal orifice at POD 7. Only 1 control fistula tract was mainly closed at POD 7. However, all control fistulas had a peripheral tract inflammation until POD 7. These results are globally similar to those previously published for the development of the model of perianal fistulizing Crohn's disease [6].

### Fistulas Treated With the Aqueous Solution of Iron Oxide Nanoparticles

As presented in Table 3, clinical examination of treated fistulas showed that all external and internal orifices were closed by USPIO at POD 7. MRI findings are shown in Table 4. MRI assessment showed that all treated fistula tracts were permanently filled or closed with USPIO up to POD 7. All external and internal orifices were closed until POD 7. Treated tracts diameters were overestimated by MRI due to the blooming effect caused by the presence of iron oxide nanoparticles. The observed diameters seemed to decrease over time suggesting metabolism of nanoparticles. Peripheral tract inflammation could not be measured because of nanoparticles' artefact. As shown in Table 5, same results with USPIO were observed until POD 14.

### Safety

Throughout our preclinical study, no rat died and all rats maintained a good general condition without any weight loss. The USPIO administration was very simple and did not require any invasive procedure. 1 rat treated with the ASION developed an abscess at POD 1 that spontaneously healed in 1 week. No other adverse effect was reported. Because liver is usually the dominant organ for clearance of the iron oxide nanoparticles [13,14], we evaluated liver function (transaminases, alkaline phosphatases and bilirubin) and made iron tests at POD 7. All biological results were in normal range. Same results were observed at POD 14 with the 2 rats treated by the ASION on the single tract.

### Conclusion

This study showed that our preclinical model of perianal fistula with peripheral inflammation was reproducible to test and optimize treatments. All treated inflammatory fistulas were permanently filled or closed with the USPIO (from day 1 to day 7), whereas only 14% were closed in the control group at day 1. A solution of iron oxide nanoparticles seems to be a promising new treatment of perianal fistulizing Crohn's disease.

### Tables

TABLE 1
MRI acquisition parameters

| | T1-weighted | T2-weighted | UTE | DWI |
|---|---|---|---|---|
| Echo time (ms) | 3.8 | 56 | 0.008 | 23 |
| Repetition time (ms) | 460 | 5300 | 4 | 2000 |
| Number of averages | 2 | 3 | 1 | 1 |
| Other specific | FLASH | RARE | 3D acquisition | directions; b values = 0, |
| parameters | sequence | sequence | | 20 segments; 3 |
| | | | | 150, 400, 800 s/mm² |
| Field of view (mm) | 60 × 60 | 60 × 60 | 60 × 60 × 60 | 60 × 60 |
| Matrix | 256 × 256 | 256 × 256 | 128 × 128 × 128 | 128 × 128 |
| Slice thickness (mm) | 1 | 1 | — | 1 |
| Number of slices | 29 | 29 | — | 11 |
| Fat saturation | Yes | Yes | No | Yes |
| Acquisition time | 3 min 55 s | 8 min 29 s | 3 min 25 s | 6 min 40 s |

TABLE 2
Clinical and radiological characteristics of the fistulas before
treatment of the 7 rats that preserved 2 fistula tracts.

| | |
|---|---|
| Treated fistula | Control fistula |

Clinical characteristics before treatment

| | | | | |
|---|---|---|---|---|
| External orifice presence | 17 | (100)^{a} | 17 | (100) |
| Internal orifice presence | 17 | (100) | 17 | (100) |
| Rectitis | | | | |

| | |
|---|---|
| Soft stools | 17 (100) |
| Perineal tumefaction | 17 (100) |
| Weight (g) | 395 ± 51 [325-521]^{b} |

MRI characteristics before treatment

| | | | | |
|---|---|---|---|---|
| Fistula tract^{c} | 17 | (100) | 17 | (100) |
| External orifice presence | 17 | (100) | 17 | (100) |
| Internal orifice presence | 16 | (94) | 17 | (100) |
| Fistula tract diameter (mm) | 2.2 ± 0.6 | [1.6-3.3] | 1.9 ± 0.3 | [1.5-2.3] |
| Peripheral tract inflammation | | | | |
| T2 Signal (a.u) | 3.60 ± 0.56 | [2.22-4.56] | 3.50 ± 0.52 | [2.13-4.61] |
| ADC (mm²/s) | 1.42 ± 0.20 | [1.058-1.709] | 1.49 ± 0.23 | [1.09-1.92] |

^{a}Number of cases (percentage of cases)
^{b}Mean ± standard deviation [range]
^{c}Fistula tract: number of rats with more than 50% of visibility of the fistula tract

TABLE 3
Clinical evolution of fistula tracts

| | |
|---|---|
| Treated fistula | Control fistula |

External orifice closed

| | | |
|---|---|---|
| J1 | 17 (100)^{a} | 3 (17) |
| J4 | 17 (100) | 15 (88) |
| J7 | 17 (100) | 15 (88) |

Internal orifice closed

| | | |
|---|---|---|
| J1 | 17 (100) | 17 (100) |
| J4 | 17 (100) | 17 (100) |
| J7 | 17 (100) | 17 (100) |

Weight (g)

| | |
|---|---|
| J1 | 395 ± 51 [325-521]^{b} |
| J4 | 407 ± 53 [329-534] |
| J7 | 414 ± 54 [337-538] |

^{a}Number of cases (percentage of cases)
^{b}Mean ± standard deviation [range]

TABLE 4
MRI findings of the fistulas after treatment of
the 7 rats that preserved 2 fistula tracts.

| | |
|---|---|
| Treated fistula | Control fistula |

POD 1

| External orifice closure | 17 | (100)^{a} | 3 | (17) |
|---|---|---|---|---|
| Internal orifice closure | 17 | (100) | 0 | |
| Filling/closing of fistula tract^{b} | 17 | (100) | 0 | |
| Fistula tract diameter (mm) | 3.7 ± 0.9 | [2.2-5.4]^{c} | 1.8 ± 0.5 | [1.2-2.9] |

| | | | |
|---|---|---|---|
| Peripheral tract inflammation | | | |
| T2 Signal (a.u) | NM | 3.14 ± 0.5 | [2.4-4.29] |
| ADC (mm²/s) | NM | 1.54 ± 0.19 | [1.20-1.94] |

POD 4

| | | | | |
|---|---|---|---|---|
| External orifice closure | 17 | (100) | 7 | (41) |
| Internal orifice closure | 17 | (100) | 2 | (11) |
| Filling/closing of fistula tract | 17 | (100) | 3 | (17) |
| Fistula tract diameter (mm) | 3.2 ± 0.8 | [1.9-4.5] | 1.6 ± 0.5 | [1.1-2.4] |

| | | | |
|---|---|---|---|
| Peripheral tract inflammation | | | |
| T2 Signal (a.u) | NM | 2.49 ± 0.5 | [1.79-3.46] |
| ADC (mm²/s) | NM | 1.55 ± 0.29 | [1.21-2.36] |

POD 7

| | | | | |
|---|---|---|---|---|
| External orifice closure | 17 | (100) | 9 | (53) |
| Internal orifice closure | 17 | (100) | 4 | (23) |
| Filling/closing of fistula tract | 17 | (100) | 5 | (29) |
| Fistula tract diameter (mm) | 3.2 ± 0.9 | [1.2-4.8] | 1.4 ± 0.3 | [0.9-2] |
| Peripheral tract inflammation | | | | |

| | | | |
|---|---|---|---|
| T2 Signal (a.u) | NM | 2.25 ± 0.5 | [1.64-3.35] |
| ADC (mm²/s) | NM | 1.49 ± 0.27 | [1.14-1.96] |

^{a}Number of cases (percentage of cases)
^{b}Filling/closing of fistula tract: number of fistula tracts filled or closed ≥50% by either ASION or natural healing.
^{c}Mean ± standard deviation [range
NM: non-measurable,
POD: post-operative day

TABLE 5
Radiological characteristics before treatment of the 3 rats with
only 1 fistula tract and MRI findings at POD 7 and POD 14.

| | | |
|---|---|---|
| ASION rats | (n = 2) | Control rat (n = 1) |

MRI characteristics before treatment

| | | | |
|---|---|---|---|
| Fistula tract^{a} | 2 | (100)^{b} | 1 (100) |
| External orifice presence | 2 | (100) | 1 (100) |
| Internal orifice presence | 2 | (100) | 1 (100) |
| Fistula tract diameter (mm) | 1.3 | [0.9-1.7]^{c} | 1.9 |
| Peripheral tract inflammation | | | |
| T2 Signal (a.u) | 4.96 | [3.96-5.96] | 3.37 |
| ADC (mm²/s) | 1.10 | [0.91-1.28] | 1.40 |

POD 7

| | | | |
|---|---|---|---|
| External orifice closure | 2 | (100) | 0 |
| Internal orifice closure | 2 | (100) | 0 |
| Filling/closing of fistula tract^{d} | 2 | (100) | 0 |
| Fistula tract diameter (mm) | 4.95 | [4.7-5.2] | 2.3 |

| | | |
|---|---|---|
| Peripheral tract inflammation | | |
| T2 Signal (a.u) | NM | 3.17 |
| ADC (mm²/s) | NM | 0.997 |

POD 14

| | | | |
|---|---|---|---|
| External orifice closure | 2 | (100) | 0 |
| Internal orifice closure | 2 | (100) | 0 |
| Filling/closing of fistula tract | 2 | (100) | 0 |
| Fistula tract diameter (mm) | 4.15 | [3.8-4.5] | 4 |

| | | |
|---|---|---|
| Peripheral tract inflammation | | |
| T2 Signal (a.u) | NM | 2.66 |
| ADC (mm²/s) | NM | 1.39 |

^{a}Fistula tract: number of rats with more than 50% of visibility of the fistula tract
^{b}Number of cases (percentage of cases)
^{c}Mean [observed values]

^{d}Filling/closing of fistula tract: number of fistula tracts filled or closed ≥50% by either ASION or natural healing.

NM: non-measurable, POD: post-operative day, ASION rats: 2 rats with only 1 fistula tract treated by the ASION, Control rat: 1 rat with only 1 fistula tract treated by a saline solution.

### REFERENCES

Throughout this application, various references describe the state of the art to which this invention pertains. The disclosures of these references are hereby incorporated by reference into the present disclosure.

[1] Sandborn W J, Fazio V W, Feagan B G, Hanauer S B, American Gastroenterological Association Clinical Practice Committee. AGA technical review on perianal Crohn's disease. Gastroenterology 2003; 125:1508-30.

[2] Bemelman W A, Warusavitarne J, Sampietro G M, Serclova Z, Zmora O, Luglio G, et al. ECCO-ESCP Consensus on Surgery for Crohn's Disease. J Crohns Colitis 2018; 12:1-16.

[3] Bouchard D, Abramowitz L, Bouguen G, Brochard C, Dabadie A, de Parades V, et al. Anoperineal lesions in Crohn's disease: French recommendations for clinical practice. Tech Coloproctology 2017; 21:683-91.

[4] Panés J, García-Olmo D, Van Assche G, Colombel J F, Reinisch W, Baumgart D C, et al. Expanded allogeneic adipose-derived mesenchymal stem cells (Cx601) for complex perianal fistulas in Crohn's disease: a phase 3 randomised, double-blind controlled trial. Lancet Lond Engl 2016; 388:1281-90.

[5] Panés J, García-Olmo D, Van Assche G, Colombel J F, Reinisch W, Baumgart D C, et al. Long-term Efficacy and Safety of Stem Cell Therapy (Cx601) for Complex Perianal Fistulas in Patients With Crohn's Disease. Gastroenterology 2018; 154:1334-1342.

[6] Flacs M, Collard M, Doblas S, Zappa M, Cazals-Hatem D, Maggiori L, et al. Preclinical Model of Perianal Fistulizing Crohn's Disease. Inflamm Bowel Dis 2019.

[7] Meddahi-Pellé A, Legrand A, Marcellan A, Louedec L, Letourneur D, Leibler L. Organ Repair, Hemostasis, and In Vivo Bonding of Medical Devices by Aqueous Solutions of Nanoparticles. Angew Chem Int Ed Engl 2014; 53:6369-73.

[8] Hyafil F, Laissy J-P, Mazighi M, Tchétché D, Louedec L, Adle-Biassette H, et al. Ferumoxtran-10-enhanced MRI of the hypercholesterolemic rabbit aorta: relationship between signal loss and macrophage infiltration. Arterioscler Thromb Vasc Biol 2006; 26:176-81.

[9] Smits L P, Tiessens F, Zheng K H, Stroes E S, Nederveen A J, Coolen B F. Evaluation of ultrasmall superparamagnetic iron-oxide (USPIO) enhanced MRI with ferumoxytol to quantify arterial wall inflammation. Atherosclerosis 2017; 263:211-8.

[10] Zheng K H, Schoormans J, Stiekema L C A, Calcagno C, Cicha I, Alexiou C, et al. Plaque Permeability Assessed With DCE-MRI Associates With USPIO Uptake in Patients With Peripheral Artery Disease. JACC Cardiovasc Imaging 2019; 12:2081-3.

[11] Plan Sangnier A, Van de Walle A B, Curcio A, Le Borgne R, Motte L, Lalatonne Y, et al. Impact of magnetic nanoparticle surface coating on their long-term intracellular biodegradation in stem cells. Nanoscale 2019; 11:16488-98.

[12] Richard S, Eder V, Caputo G, Journé C, Ou P, Bolley J, et al. USPIO size control through microwave nonaqueous sol-gel method for neoangiogenesis T2 MRI contrast agent. Nanomed 2016; 11:2769-79.

[13] Lee M J-E, Veiseh O, Bhattarai N, Sun C, Hansen S J, Ditzler S, et al. Rapid pharmacokinetic and biodistribution studies using cholorotoxin-conjugated iron oxide nanoparticles: a novel non-radioactive method. PloS One 2010.

[14] Arami H, Khandhar A, Liggitt D, Krishnan K M. In vivo delivery, pharmacokinetics, biodistribution and toxicity of iron oxide nanoparticles. Chem Soc Rev 2015; 44:8576-607.

1. A method of treating a fistulizing anoperineal lesion in patient in need thereof comprising injecting in the fistula tract a solution comprising an amount of nanoparticles.

2. The method of claim 1 wherein the patient suffers from an inflammatory bowel disease.

3. The method of claim 2 wherein the patient suffers from Crohn's disease.

4. The method of claim 1 wherein the nanoparticles have a size of 5, 10, 15 or 20 nm.

5. The method of claim 1 wherein the nanoparticles are made of at least one metal oxides.

6. The method of claim 5 wherein the nanoparticles are iron oxide Fe₃O₄ Ultrasmall Superparamagnetic Iron Oxide (USPIO) nanoparticles.

7. The method of claim 1 wherein the concentration of the nanoparticles is comprised between 10 mg/ml and 1000 mg/ml.

8. The method of claim 1 wherein once the solution of nanoparticles is injected in the fistula tract, a pressure is applied on the surface where the fistula tract arises.

9. The method of claim 8 wherein the pressure is applied on the peritoneum.

10. The method of claim 8 wherein the pressure is applied for a time of at least 60 s.

11. The method of claim 1 wherein the nanoparticles are injected in the fistula tract as an aqueous solution.

12. The method of claim 1 wherein the nanoparticles are injected in the fistula tract as an aqueous alcohol solution of nanoparticles.

13. The method of claim 1 wherein the nanoparticles are injected in combination with a TNFα blocking agent.

14. The method of claim 5 wherein the at least one metal oxide is an iron oxide, cerium oxide (CeO), aluminum oxide (Al₂O₃), zirconium oxide (ZrO₂), titanium oxide (TiO₂), a titanate, indium oxide (In₂O₃), tin oxide (SnO₂), antimony oxide (Sb₂O₃), magnesium oxide (MgO), calcium oxide (CaO), a manganese oxide, molybdenum oxide (MoO₃), silica (SiO₂), zinc oxide (ZnO), yttrium oxide (Y₂O₃), bismuth oxychloride, or a copper oxides (CuO, Cu₂O).

15. The method of claim 14, wherein: the iron oxide is FeO, Fe₂O₃ or Fe₃O₄; the titanate is BaTiO₃, Ba_{0.5}Sr_{0.5}TiO or SrTiO₃; the manganese oxide is Mn₃O₄ or MnO₂; and the copper oxide is CuO or Cu₂O.
